(19) United States
(12) Patent Application Publication
(10) Pub. No.: US 2016/0158551 A1
(43) Pub. Date: Jun. 9, 2016

(54) SYSTEM AND METHOD FOR DORSAL ROOT BLOCK DURING SPINAL CORD STIMULATION

(71) Applicant: Pacesstter, Inc., Sunnyvale, CA (US)

(72) Inventors: Alexander Kent, Mountain View, CA (US); Xiaoyi Min, Camarillo, CA (US)

(21) Appl. No.: 14/562,251

(22) Filed: Dec. 5, 2014

(51) Int. Cl. A61N 1/36

(52) U.S. Cl. CPC A61N 1/36139; A61N 1/36071

(57) ABSTRACT

A system and method are provided to mitigate excitation of non-target regions of nerve fibers during spinal cord stimulation (SCS) of nervous tissue of a patient. The method and system deliver an SCS excitation waveform to an excitation electrode located proximate to a dorsal column (DC), the SCS excitation waveform shaped to excite a nerve fiber target region (TR) within the DC. The method and system also deliver a blocking waveform to a blocking electrode located proximate to the DR, the excitation waveform shaped to at least partially induce hyperpolarization into a nerve fiber non-target region (NTR) within the DR.

### BACKGROUND OF THE INVENTION

Embodiments of the present disclosure generally relate to neurostimulation (NS), and more particularly to blocking excitation of non-target regions of nerve fibers.

Spinal cord stimulation (SCS) is used to treat a wide range of chronic neuropathic pain conditions by delivering electrical stimulation to select portions of the spinal cord. In the past, SCS therapy has been proposed in which a tonic therapy is defined by single pulses having a select pulse width, frequency and intensity. By way of example, tonic therapies have been proposed to manage cervical and lumbar pain. The pulse width, frequency and intensity may be changed, along with electrode configuration and placement on the spinal column in connection with pain relief for individual patients.

NS systems are devices that generate electrical pulses and deliver the pulses to nervous tissue to treat a variety of disorders. For example, spinal cord stimulation has been used to treat chronic and intractable pain. Another example is deep brain stimulation, which has been used to treat movement disorders such as Parkinson's disease and affective disorders such as depression. Application of electrical pulses to certain regions or areas of nervous tissue can effectively alter or reduce the number of pain signals that reach the brain. For example, applying electrical energy to the spinal cord associated with regions of the body afflicted with chronic pain can induce “paresthesia” (a subjective sensation of numbness or tingling) in the afflicted bodily regions.

SCS therapy, delivered via epidurally implanted electrodes, is a widely used treatment for chronic intractable neuropathic pain of different origins. During SCS therapy calibration, the paresthesia is identified and localized to the painful areas by the patient in connection with determining correct electrode placement.

In spinal cord stimulation (SCS), nerve fibers in the dorsal column (DC) are targeted for excitation, but unintended excitation of non-targeted fibers, such as in the dorsal roots (DRs) may also occur. Activation of DC nerve fibers produces coincident paresthesia and pain relief in the corresponding body dermatomes that the target nerve fibers innervate. Conversely, activation of DR nerve fibers can produce uncomfortable sensations or other side effects.

During each stimulation pulse, current travels from the positive, anodic contact(s) (electrodes) to the negative, cathodic contact(s) (electrodes). Nerve fibers that are located near the cathodic electrode are depolarized (i.e., the transmembrane potential becomes less negative), and sufficient depolarization generates action potentials (APs). On the other hand, fibers near the anodic electrode are hyperpolarized (i.e., the transmembrane potential becomes more negative). Even when an AP is already initiated in a given nerve fiber, sufficient hyperpolarization of the nerve fiber at a downstream location may block AP propagation.

In the past, leads utilized an “anodal guarding” configuration that sets the electrodes on the outer column(s) of a paddle SCS lead to anodes, thereby attempting to guard the DR fibers from the medially-located cathodic electrode(s). However, the anodal guarding configuration is not widely used in the clinical setting. Computational modeling indicates that anodal guarding does not improve the relative activation thresholds for DR or DC nerve fibers.

### SUMMARY

In accordance with embodiments, a method is provided to mitigate excitation of non-target regions of nerve fibers during spinal cord stimulation (SCS) of nervous tissue of a patient. The method comprises delivering an SCS excitation waveform to an excitation electrode located proximate to a dorsal column (DC), the SCS excitation waveform shaped to excite a nerve fiber target region (TR) within the DC. The method also delivers a blocking waveform to a blocking electrode located proximate to the DR, the blocking waveform shaped to at least partially induce hyperpolarization into a nerve fiber non-target region (NTR) within the DR. The method also manages the delivering operations to at least one of: time delivery of the SCS excitation and blocking waveforms to at least partially overlap, adjusting a timing of the blocking waveform based on a propagation speed at which action potentials propagate along the nerve fibers, or shaping the blocking waveform with at least one pulse having a trailing edge with a non-zero slope.

Optionally, the excitation electrode may be positioned proximate to the nerve fiber TR within the DC, wherein the blocking electrode is positioned proximate to the nerve fiber NTR within the DR. Optionally, the blocking waveform may have at least one of a trapezoidal shape or non-rectangular shape. Alternatively, the blocking waveforms may induce hyperpolarization into at least one of A-delta fibers, A-beta fibers or C-fibers within the NT-NTR. Optionally, the method may further comprise positioning the at least one blocking electrode proximate to at least one of the dorsal root or dorsal root ganglion. Alternatively, the managing operation may include timing a delivery of pulses in the blocking waveform to at least one of selectively prevent excitation or impede action potential propagation in nerve fibers within the DR corresponding to the nerve fiber NTR.

Optionally, the delivering operation may include generating multiple blocking pulses in the blocking waveform in connection with a single SCS pulse in the SCS excitation waveform, the blocking pulses timed to at least partially overlap the SCS pulse. Alternatively, the SCS excitation and blocking waveforms may include an SCS pulse and blocking pulse, respectively, the delivery operation timing the blocking pulse to start earlier or end later than the SCS pulse. Optionally, the blocking waveform may be configured to impede action potential propagation in nerve fibers within the DR in the afferent direction, thereby reducing side effects of the excitation waveform.

Alternatively, the blocking waveform may be configured to hyperpolarize the nerve fibers in the DR near AP initiation. Optionally, the method may further comprise delivering recharge pulses following the excitation and blocking waveforms, the recharge pulses configured to at least partially neutralize a charge remaining in the nerve fibers following delivery of the excitation and blocking waveforms. The method may further comprise measuring an action potential (AP) signal to obtain AP activity data for the nerve fiber NTR, deriving the propagation speed of the nerve fiber NTR based on the AP activity data, and adjusting the timing of the blocking waveform based on the propagation speed at which action potentials propagate along the nerve fibers.

In accordance with embodiments, a system is provided to mitigate excitation of non-target regions of nerve fibers during spinal cord stimulation (SCS) of nervous tissue of a patient. The system comprises a lead having an excitation electrode that is configured to be located proximate to a dorsal column (DC) and having a blocking electrode configured to be located proximate to the DR. The system also comprises a processor, a pulse generator and memory storing program instructions accessible by the processor. Responsive to execution of the program instructions, the processor delivers a SCS excitation waveform to the excitation electrode, the SCS excitation waveform shaped to excite a nerve fiber target region (TR) within the DC. The processor also delivers a blocking waveform to a blocking electrode located proximate to the DR, the blocking waveform shaped to at least partially induce hyperpolarization into a nerve fiber non-target region (NTR) within the DR. The processor also manages the delivering operations to at least one of: time delivery of the SCS excitation and blocking waveforms to at least partially overlap; adjust a timing of the blocking waveform based on a propagation speed at which action potentials propagate along the nerve fibers; or shape the blocking waveform with at least one pulse having a trailing edge with a non-zero slope.

Optionally, the excitation electrode may be positioned proximate to the nerve fiber TR within the DC, wherein the blocking electrode is positioned proximate to the nerve fiber NTR within the DR. Optionally, the pulse generator may further comprise first and second current sources that independently deliver the SCS excitation waveform and blocking waveform, respectively. Optionally, the pulse generator may shape a pulse of the blocking waveform to have a tapered trailing average with a nonzero slope when transitioning between high and low levels.

Optionally, the lead may include an array of electrodes including the excitation and blocking electrodes. Optionally, the processor may couple the blocking electrode to an anode of the pulse generator, that the blocking electrode formed an anodic electrode when delivering the blocking waveform, the system further comprising an implantable medical device having a housing coupled to the lead, the processor coupling the housing of the IPG to a cathode of the pulse generator such that the housing forms a cathodic electrode when delivering the blocking waveform.

Optionally, the lead may include an array of electrodes including the blocking electrode, the blocking electrode having an anodic polarity when delivering the blocking waveform, the lead excluding any electrodes with a cathodic polarity in connection with delivering the blocking waveform. Optionally, the managing operation performed by the processor may include timing a delivery of pulses in the blocking waveform to at least one of selectively prevent excitation or impede action potential propagation in nerve fibers within the DR corresponding to the nerve fiber NTR.

### BRIEF DESCRIPTION OF THE DRAWINGS

FIG. 1 illustrates a neurostimulation system that generates electrical pulses for application to tissue of a patient, according to an embodiment of the present disclosure.

FIGS. 2A-2E respectively depict stimulation portions for inclusion at the distal end of lead in accordance with embodiments herein.

FIG. 3 illustrates a graphical representation of a spinal cord and dorsal root fibers joined thereto, in accordance with embodiments herein.

FIGS. 4A and 4B illustrate a paddle lead positioned in the dorsal epidural space in the spinal canal in accordance with embodiments herein.

FIG. 5 illustrates a process to mitigate excitation of non-target regions of nerve fibers during spinal cord stimulation of nervous tissue of a patient in accordance with embodiments herein.

FIGS. 6A and 6B illustrate examples of stimulation and blocking pulse delivery timing provided in accordance with an embodiment herein, in order to afford anodal blocking of propagation of action potentials within the dorsal root in accordance with embodiments herein.

FIG. 7 illustrates a functional block diagram of an embodiment of an electronic control unit (ECU) that is operated in accordance with the processes described herein.

### DETAILED DESCRIPTION

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

In accordance with embodiments herein, methods and systems are provided that time delivery of spinal cord stimulation (SCS) and blocking pulses to selectively prevent excitation or impede action potential (AP) propagation in dorsal root (DR) fibers. The blocking pulse is configured to reduce side effects associated with DR stimulation, and allow better localization of excitation to targeted fibers in the dorsal column (DC). Multiple pulses are delivered sequentially, including an excitation pulse, blocking pulse(s), and recharge pulses. The excitation pulse is delivered from clinically therapeutic electrodes to activate targeted fibers in the DC, but may also excite non-targeted fibers in the DR. The blocking pulse(s) are delivered from anodal electrodes located near the DR fibers to selectively block the non-target fibers. Recharge pulses are delivered to maintain charge balance. Alternatively, recharge pulses may be combined with stimulation pulses. The excitation and blocking pulses may be delivered concurrently, with either pulse starting earlier or ending later than the other. The stimulation parameters (e.g. amplitude, pulse width, delay, waveform shape, electrode configuration) used for the pulses are flexible and can be adjusted based on electrophysiological properties of nerve fibers.

### Nervous System Overview

Embodiments herein utilize different configurations of blocking pulses (e.g., electrode configuration/position, number of pulses, pulse width, amplitude, shape, pulse timing) to hyperpolarize select types of fibers (e.g., A-beta, A-delta, C-fibers) at select areas along the dorsal root (e.g., proximate select vertebra). Accordingly, it is useful to understand differences in the fiber structure, AP propagation speeds and nature of the sensations conveyed by the various fibers along the DR and DRG.

The nervous system is comprised of the central nervous system (CNS) and the peripheral nervous system (PNS). The CNS contains the brain and spinal cord. The PNS is comprised mainly of mixed nerves, which are enclosed bundles of the long fibers or axons (endings of nerve cells or neurons) that connect the CNS to every other part of the body. The types of nerve fibers in a mixed nerve include autonomic nerve fibers, sensory nerve fibers (afferent fibers sending information towards the brain) and motor nerve fibers (efferent fibers sending information from the brain). The autonomic nerve fibers innervate smooth muscles, cardiac muscle, and glands. Sensory neurons transmit information from the environment, such as pain, and motor neurons mediate voluntary and involuntary movement.

In general, the peripheral nerve fibers may be classified into three types of nerve fibers based on the nerve fiber diameter and conduction velocity, namely A-, B- and C-fibers. A-fibers have large diameters, high conduction velocities, are highly myelinated, and are further subdivided by size and conduction velocity as A-alpha, A-beta, A-gamma and A-delta fibers. By way of example, the fast conduction velocity of the A-alpha fibers may be on the order of 80-120 m/s, and the A-alpha fibers may be on average 13-20 μm in diameter. B-fibers have diameters of about 3 um and conduction velocities of 3-15 m/s. C-fibers are small neurons with slow conduction velocities and are not myelinated.

A-delta fibers have conduction velocities on the order of 5-35 m/s, and the A-delta fibers may be on average 1.0-5.0 μm in diameter. A-delta fibers carry information mainly from the nociceptive-mechanical or mechanothermal-specific stimuli and are considered nociceptors. The receptive fields (area of innervation) are small, and therefore, provide precise localization of pain.

C-fibers are unmyelinated, have a small diameter and low conduction velocity. By way of example, the slow conduction velocity of the C-fibers may be on the order of 0.5-2.0 m/s, and the C-fibers may be on average 0.2-1.5 μm in diameter. C-fibers carry sensory information, such as nociception (pain), temperature, and itch. C-fibers are unmyelinated unlike most other fibers in the nervous system. The lack of myelination is, at least in part, a cause of the slow conduction velocity attributed to C-fibers. C-fibers are activated by and carry information from a variety of high-intensity mechanical, chemical and thermal stimulation and thus are considered as polymodal nociceptors.

The cell bodies of all primary afferent pain neurons from the body, face, and head are located in the dorsal root ganglia (DRG) and in the trigeminal ganglia respectively. Some of these cell bodies have myelinated axons (A-delta fibers), and others have unmyelinated axons (C-fibers). Both A-delta fiber's and the unmyelinated C-fiber's axons have free nerve endings, which innervate the same areas in the periphery. A-delta fibers are responsible for the sensation of a quick shallow pain that is specific on one area, termed as first pain. The A-delta fibers respond to a weaker intensity of noxious stimulus. C-fibers respond to noxious stimuli which have stronger intensities and account for the slow, but deeper second pain that spreads out over an unspecific area.

When the blocking waveform is directed primarily at APs in the A-delta fibers, a first timing for the blocking waveform may be used based on the conduction velocity in A-delta fibers. A different second timing may be used for the blocking waveform when directed to C-fibers. The first and second timing may represent an offset between leading edges of the excitation and blocking waveforms.

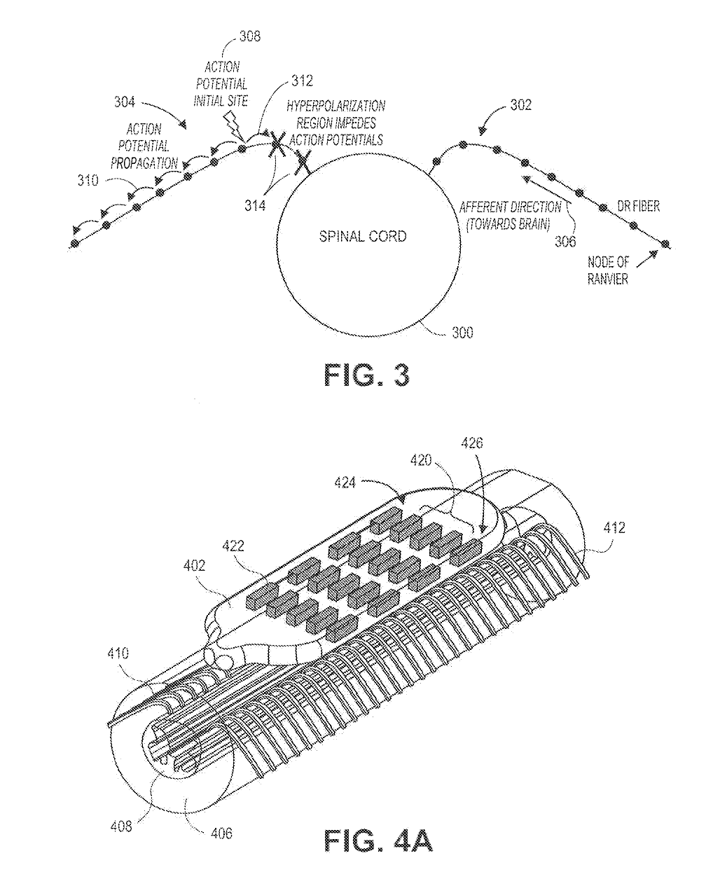

FIG. 3 illustrates a graphical representation of a spinal cord 300 and dorsal root fibers 302, 304 joined thereto. Action potentials propagate along the DR fibers 302 and 304 and the afferent direction as denoted at 306 toward the spinal cord 300 and the brain. When an SCS therapy is delivered at nerve fibers in a target region near the dorsal column of the spinal cord 300, a potential exists that such SCS therapy may be experienced at a site of initiation 308 along the nerve fibers (referred to herein as non-target regions). When an SCS therapy is experienced at a nerve fiber in a non-target region, such as at the site of initiation 308, the SCS therapy may induce an action potential that propagates in both directions along the affected nerve fiber from one node of the Ranvier to the next node (e.g., salutary conduction) as denoted by arrows 310 and 312. Arrows 310 indicate that action potentials may propagate in an efferent direction, namely in a direction away from the brain that is opposite to the afferent direction. To prevent the action potential to propagate from the initiation site 308 toward the brain, in accordance with embodiments herein, a blocking waveform is delivered at the nerve fiber non-target region denoted at 314 (corresponding to the dorsal root or dorsal root ganglia). In accordance with embodiments herein, the blocking waveform induces hyperpolarization at the affected nerve fiber non-target region to impede propagation of the action potentials.

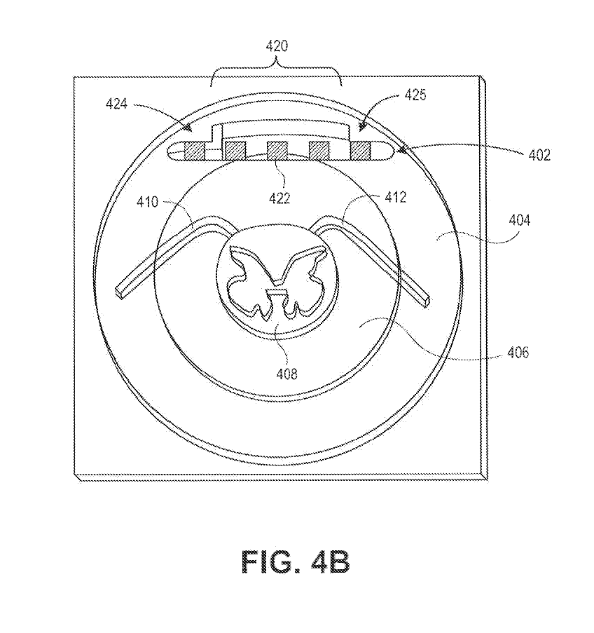

FIGS. 4A and 4B illustrate a paddle lead 402 positioned in the dorsal epidural space in the spinal canal in accordance with embodiments herein. FIG. 4A illustrates the lead 402 from a dorsal lateral angle, while FIG. 4B illustrates the lead 402 from a transverse cross-sectional view. As illustrated in FIG. 4B, the lead 402 is positioned within the epidural fatty layer 404 which is located proximate to the cerebral spinal fluid layer 406 which surrounds the dorsal column 408. Dorsal root fibers 410, 412 extend outward from the dorsal column 408 at various points along the length thereof.

As shown in FIG. 4A, one or more central columns 420 of electrodes 422 on the lead 402 are positioned in close proximity to the dorsal column 408. Peripheral columns 424 and 426 of electrodes 422 are located on opposite sides of the central columns 420. The peripheral columns 424 and 426 are located proximate to the dorsal root fibers 410, 412. Optionally, a single column of electrodes 422 may be provided within the central column 420, while multiple columns of electrodes may be provided within the peripheral columns 424 and/or 426. As explained herein, electrodes 422 within one or more of the central columns 420 may be utilized to deliver an SCS therapy and recharge pulses, while one or more electrodes 422 within one or more of the peripheral columns 424, 426 may be used to deliver blocking waveforms and recharge pulses.

### Processes to Mitigate Excitation of Non-Target Nerve Fibers

In accordance with embodiments herein, methods and systems are described for delivering blocking waveforms to selectively impede action potential (AP) propagation in nerve fibers in non-target regions, such as associated with the dorsal roots (DRs), and thereby reduce side effects of SCS stimulation waveforms. Absent a blocking waveform, delivery of each excitation pulse to the dorsal spinal cord activates nerve fibers in the targeted dorsal column (DC), as well as nerve fibers in the non-target region associated with the DR. Embodiments herein deliver blocking waveforms (e.g. in the form of high-intensity blocking pulses) in order to hyperpolarize the nerve fibers in the non-target region associated with the DR, which also corresponds to the location near the site of AP initiation. Sufficient hyperpolarization of nerve fibers in the non-target region blocks action potential propagation in the afferent direction (e.g. towards the brain). Consequently, an AP signal does not continue to propagate along the spinal cord towards the brain, and little or no sensations or side effects are experienced by the patient.

In accordance with embodiments herein, the blocking waveform may be delivered by laterally-positioned “anodal” electrodes located close to the DRs. For example, electrodes on the outer columns of a paddle lead may be used to deliver the blocking waveform. Additionally or alternatively, multiple percutaneous leads may be implanted in an adjacent arrangement along the medial-lateral direction, such that the electrodes on the laterally-positioned leads are defined as anodal electrodes and used to deliver the blocking waveform. The blocking pulse can be directed at DRs on one side (unilateral) or both sides (bilateral), using electrodes positioned over the left and/or right DRs. The option to use unilateral or bilateral blocking waveforms may depend in part on the location of electrodes used to deliver the excitation waveform and corresponding area of activation. Alternatively, the blocking waveform may be delivered from a lead that is distinct from the lead used for delivery of the excitation waveform. For example, a separate lead may be placed over the DR or dorsal root ganglia in order to deliver blocking pulses. Utilizing a separate lead to deliver the blocking waveform may decrease the current levels utilized for blocking because the dedicated blocking lead is positioned close to the DRs, and positioned with more specificity for blocking DR fibers.

The SCS stimulation and blocking waveforms are defined by one or more parameters forming a therapy parameter set (TPS) and a mitigation parameter set (MPS). The parameters for the TPS include a selection of which electrodes on the lead(s) are utilized to deliver the SCS stimulation waveform. The parameters for the MPS include a selection of which electrodes on the lead(s) are utilized to deliver the blocking waveform. The parameters for the TPS and/or the MPS also include a selection of a polarity (e.g., anode or cathode) to be assigned to each electrode to be used when delivering the SCS stimulation waveform, and a polarity to be assigned to each electrode to be used when delivering the blocking waveform. Examples of other parameters in the TPS and/or MPS include pulse amplitude, pulse width, pulse frequency or inter-pulse period, pulse repetition parameter (e.g., number of times for a given pulse to be repeated for respective stimset during execution of program), waveform shape (e.g., biphasic pulses, monophasic pulses), etc.

The terms “anodic”, “anodal” and “anode” refer to an electrode through which positive electric charge flows into the electrode. Electric current flows from the anode to the cathode. Since electrons flow in the direction opposite of electric current, they flow out of the cathode to the anode. The terms “cathodic”, “cathodal” and “cathode” refer to an electrode through which positive electric charge flows out of the electrode and into the tissue or nerve fiber of interest. It is recognized that the direction of flow of electric current is opposite to the direction of electron flow, where electrons flow out of the anode electrode(s) and where electrons flow into the cathode electrode(s). The cathode has a negative polarity with respect to the anode. Positively charged cations move towards the cathode and negatively charged anions move away from away from the cathode.

### System Overview

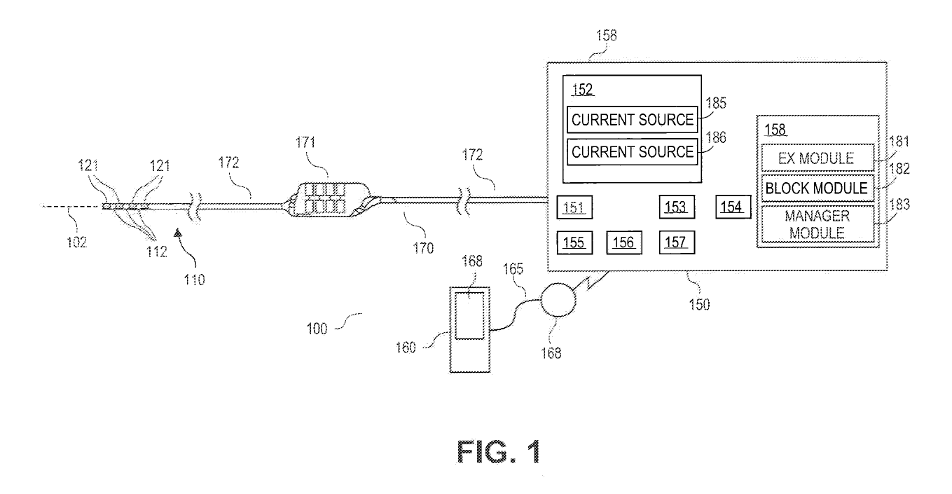

FIG. 1 depicts an NS system 100 that generates electrical pulses for application to tissue of a patient according to one embodiment. For example, the NS system 100 may be adapted to stimulate spinal cord tissue, peripheral nervous tissue, deep brain tissue, cortical tissue, cardiac tissue, digestive tissue, pelvic floor tissue, or any other suitable nervous tissue of interest within a patient's body.

The NS system 100 may be controlled to deliver various types of non-paresthesia therapy, such as high frequency neurostimulation therapies, burst neurostimulation therapies and the like. High frequency neurostimulation includes a continuous series of monophasic or biphasic pulses that are delivered at a predetermined frequency (such as 2-10 kHz). Burst neurostimulation includes short sequences of monophasic or biphasic pulses, where each sequence is separated by a quiescent period. By way of example, the pulses within each burst sequence may be delivered with an intraburst frequency of about 500 Hz. In general, non-paresthesia therapies include a continuous, repeating or intermittent pulse sequence delivered at a frequency and amplitude configured to avoid inducing (or introduce a very limited) paresthesia.

The NS system 100 may continuously or repeatedly deliver an SCS therapy (and blocking pulses) without sensing feedback. Optionally, the NS system 100 may represent a closed loop neurostimulation device configured to provide real-time sensing functions for A-delta and C-fiber action potential (APs) from a dorsal root ganglion (DRG) lead. The configuration of the lead sensing electrodes that sense action potentials from the A-delta and C fibers may be varied depending on the neuronal anatomy of the sensing site(s) of interest. The size and shape of electrodes is varied based on the implant location, such as the dorsal root (DR) or DRG. By way of example only, a laminectomy procedure may be used, in order to obtain accurate action potential signals indicative of pain from the C fiber and/or the A-delta fiber. The electronic components within the NS system 100 are designed with both stimulation and sensing capabilities. The NS system 100 may detect A-delta and C-fiber action potentials and utilize changes to the frequency of the A-delta and C-fiber action potential signals to guide parameter settings for the blocking waveform. In one embodiment, one lead stimulates the dorsal column, while the second lead senses from DRG or DR. In another embodiment, a common lead may be used to deliver a blocking waveform to the DRG or DR nerve fibers, followed (or preceded) by sensing of AP signals from the same electrode location(s).

The NS system 100 includes an implantable pulse generator (IPG) 150 that is adapted to generate the stimulation and blocking waveforms as electrical pulses. The IPG 150 may comprise a metallic housing or can 158 that encloses one or more controllers 151, one or more pulse generating circuitry 152, a charging coil 153, a battery 154, a far-field and/or near field communication circuitry 155, battery charging circuitry 156, switching circuitry 157, memory 158 and the like. The housing 158 may be designated as an anodic or cathodic electrode by changing the settings of the switching circuitry 157 to connect the housing 158 to a corresponding terminal (anode or cathode) of the pulse generating circuitry 152. The controller 151 typically includes one or more microcontrollers or other suitable processor for controlling the various other components of the device. Software code is typically stored in memory of the IPG 150 for execution by the microcontroller or processor to control the various components of the device.

The IPG 150 may comprise a separate or an attached extension component 170. If the extension component 170 is a separate component, the extension component 170 may connect with the “header” portion of the IPG 150 as is known in the art. If the extension component 170 is integrated with the IPG 150, internal electrical connections may be made through respective conductive components. Within the IPG 150, electrical pulses are generated by the pulse generating circuitry 152 and are provided to the switching circuitry 157. The switching circuitry 157 connects to outputs of the IPG 150. Electrical connectors (e.g., “Bal-Seal” connectors) within the connector portion 171 of the extension component 170 or within the IPG header may be employed to conduct various stimulation pulses. The terminals of one or more leads 110 are inserted within connector portion 171 or within the IPG header for electrical connection with respective connectors. Thereby, the pulses originating from the IPG 150 are provided to the lead 110. The pulses are then conducted through the conductors of the lead 110 and applied to tissue of a patient via electrodes 121 that are coupled to blocking capacitors. Any suitable known or later developed design may be employed for connector portion 171. Optionally, the extension component 170 and connector portion 171 may be omitted and the lead 110 directly connected to the header of the IPG 150.

The lead 110 may represent one or more paddle leads, percutaneous leads, or distinct leads implanted on or near the DC, DR or dorsal root ganglia. Before, during, and/or after an excitation pulse is delivered by electrodes near the DC, an anodic blocking pulse is delivered by anodal electrodes near the DR, followed by or in combination with recharge pulses.

The electrodes 121 may be positioned along a horizontal axis 102 of the lead 110, and are angularly positioned about the horizontal axis 102 so the electrodes 121 do not overlap. The electrodes 121 may be in the shape of a ring such that each stimulation electrode 121 continuously covers the circumference of the exterior surface of the lead 110. Each of the electrodes 121 are separated by non-conducting rings 112, which electrically isolate each stimulation electrode 121 from an adjacent stimulation electrode 121. The non-conducting rings 112 may include one or more insulative materials and/or biocompatible materials to allow the lead 110 to be implantable within the patient. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane. The electrodes 121 may be configured to emit the pulses in an outward radial direction proximate to or within a stimulation target. Additionally or alternatively, the electrodes 121 may be in the shape of a split or non-continuous ring such that the pulse may be directed in an outward radial direction adjacent to the electrodes 121. The electrodes 121 deliver high frequency and/or burst stimulation waveforms as described herein. The electrodes 121 may also sense sensory action potential (SAP signals) for a data collection window. Optionally, the delivering operation may deliver an SCS excitation waveform to a first sub-set of the electrodes and a blocking waveform to a second sub-set of the electrodes.

Optionally, the electrodes may include a microelectrode located immediately adjacent nerve fibers of interest. The method may sense a fiber sensory action potential (SAP) directly at the microelectrode and perform an iterative feedback loop to adjust at least one therapy or blocking parameter based on the A-delta or C-fiber SAP. For example, a leading edge of a blocking pulse may be shifted temporally earlier or later in time relative to a leading edge of the excitation pulse based on whether the SAP propagates slow or fast.

The lead 110 may comprise a lead body 172 of insulative material about a plurality of conductors within the material that extend from a proximal end of lead 110, proximate to the IPG 150, to its distal end. The conductors electrically couple a plurality of the electrodes 121 to a plurality of terminals (not shown) of the lead 110. The terminals are adapted to receive electrical pulses and the electrodes 121 are adapted to apply the pulses to the stimulation target of the patient. Also, sensing of physiological signals may occur through the electrodes 121, the conductors, and the terminals. It should be noted that although the lead 110 is depicted with four electrodes 121, the lead 110 may include any suitable number of electrodes 121 (e.g., less than four, more than four) as well as terminals, and internal conductors. Additionally or alternatively, various sensors (e.g., a position detector, a radiopaque fiducial) may be located near the distal end of the lead 110 and electrically coupled to terminals through conductors within the lead body 172. The lead 110 may represent one or more paddle leads, percutaneous leads, or distinct leads implanted on or near the DR or dorsal root ganglia. Before, during, and/or after an excitation pulse is delivered by electrodes near the DC, an anodic blocking pulse is delivered by anodal electrodes near the DR, followed by or in combination with recharge pulses.

Although not required for any embodiments, the lead body 172 of the lead 110 may be fabricated to flex and elongate upon implantation or advancing within the tissue (e.g., nervous tissue) of the patient towards the stimulation target and movements of the patient during or after implantation. By fabricating the lead body 172, according to some embodiments, the lead body 172 or a portion thereof is capable of elastic elongation under relatively low stretching forces. Also, after removal of the stretching force, the lead body 172 may be capable of resuming its original length and profile. For example, the lead body may stretch 10%, 20%, 25%, 35%, or even up or above to 50% at forces of about 0.5, 1.0, and/or 2.0 pounds of stretching force. Fabrication techniques and material characteristics for “body compliant” leads are disclosed in greater detail in U.S. Provisional Patent Application No. 60/788,518, entitled “Lead Body Manufacturing,” which is expressly incorporated herein by reference.

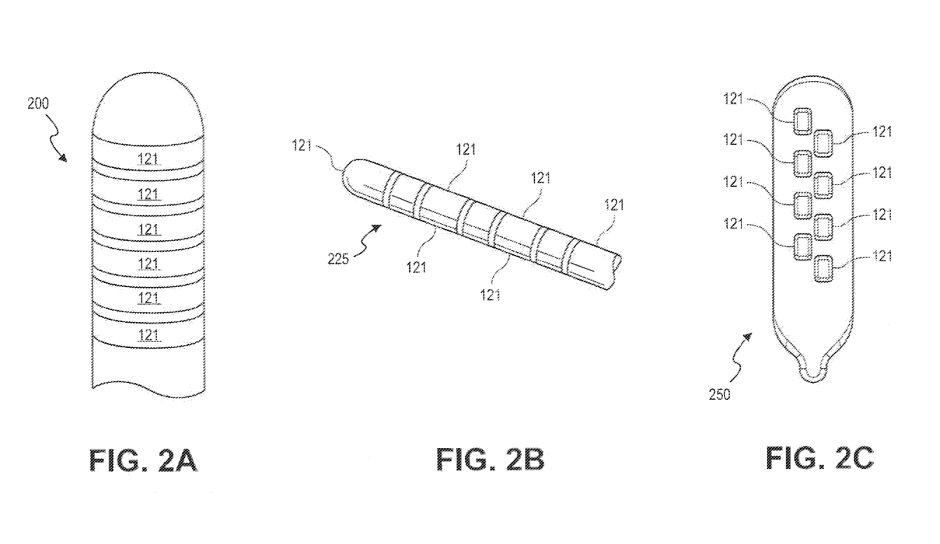

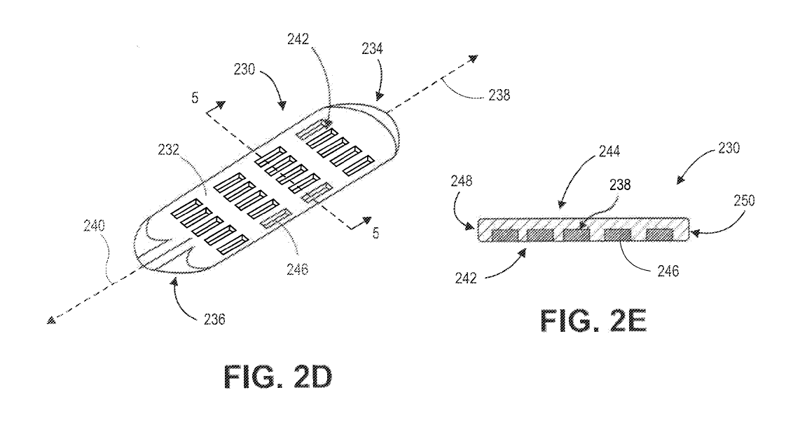

FIGS. 2A-2E respectively depict stimulation portions 200, 225, 230 and 250 for inclusion at the distal end of lead 110. Stimulation portion 200 depicts a conventional stimulation portion of a “percutaneous” lead with multiple ring electrodes. Stimulation portion 225 depicts a stimulation portion including several segmented electrodes. Example fabrication processes are disclosed in U.S. patent application Ser. No. 12/895,096, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure. Returning to FIG. 1, for implementation of the components within the IPG 150, a processor and associated charge control circuitry for an IPG is described in U.S. Pat. No. 7,571,007, entitled “SYSTEMS AND METHODS FOR USE IN PULSE GENERATION,” which is expressly incorporated herein by reference. Circuitry for recharging a rechargeable battery (e.g., battery charging circuitry 156) of an IPG using inductive coupling and external charging circuits are described in U.S. Pat. No. 7,212,110, entitled “IMPLANTABLE DEVICE AND SYSTEM FOR WIRELESS COMMUNICATION,” which is expressly incorporated herein by reference.

An example and discussion of “constant current” pulse generating circuitry (e.g., pulse generating circuitry 152) is provided in U.S. Patent Publication No. 2006/0170486 entitled “PULSE GENERATOR HAVING AN EFFICIENT FRACTIONAL VOLTAGE CONVERTER AND METHOD OF USE,” which is expressly incorporated herein by reference. Multiple sets of the pulse generating circuitry 152 may be provided within the IPG 150. The excitation and blocking pulses on different electrodes 121 are generated using separate pulse generating circuitry 152. Complex pulse parameters may be employed such as those described in U.S. Pat. No. 7,228,179, entitled “Method and apparatus for providing complex tissue stimulation patterns,” and International Patent Publication Number WO 2001/093953 A1, entitled “NEUROMODULATION THERAPY SYSTEM,” which are expressly incorporated herein by reference. Alternatively, multiple sets of such circuitry may be employed to provide different pulse patterns (e.g., tonic stimulation waveform, burst stimulation waveform) that include generated and delivered stimulation pulses through various stimulation electrodes of one or more leads 121 as is also known in the art. Various sets of parameters may define the pulse characteristics and pulse timing for the pulses applied to the various electrodes 121. Although constant current pulse generating circuitry is contemplated for some embodiments, any other suitable type of pulse generating circuitry may be employed such as constant voltage pulse generating circuitry.

The controller 151 iteratively repeats the delivering and sensing operations for a group of TPS and MPS. The controller 151 analyzes the SAP signals to obtain activity data associated with the TPS and MPS for at least one of SAP C-fiber components or SAP A-delta fiber components, the analyzing operations obtaining a collection of activity data associated with the group of TPS and MPS. The IPG selects a candidate TPS and MPS from the group of TPS and MPS based on a criteria of interest.

Memory 158 stores software to control operation of the controller 151 for mitigating excitation of non-target regions of nerve fibers during spinal cord stimulation (SCS) of nervous tissue of a patient. The memory 158 also stores SAP signals, therapy parameter sets, mitigation parameter sets, SAP activity level data, pain scales and the like. For example, the memory 158 may save SAP activity level data for various different therapies, blocking waveforms and recharge pulses. A collection of SAP activity level data is accumulated for different therapies and blocking waveforms and may be compared to identify high, low and acceptable amounts of sensory activity for the A-delta and/or C-fibers in the non-target regions. The memory 158 may store a pain-activity data relation defining a relation between content of the SAP signals and pain scores indicative of pain experienced by a patient.

The memory 158 includes an excitation module 181 that directs the controller 151 to control delivery of SCS excitation waveforms to the excitation electrode(s), where the SCS excitation waveform is shaped to excite a nerve fiber target region (TR) within the DC. The memory 158 also includes a blocking module 182 that directs the controller 151 to control delivery of blocking waveforms to a blocking electrode located proximate to the DR, the blocking waveform shaped to at least partially induce hyperpolarization into a nerve fiber non-target region (NTR) within the DR. In accordance with some embodiments, the blocking module 182, through the controller 151, directs the switching circuitry to couple the blocking electrodes on the lead to the pulse generating circuitry such that any blocking electrodes on the lead only have an anodic polarity when delivering the blocking waveform in order that the lead entirely lacks and excludes any electrodes with a cathodic polarity when delivering the blocking waveform. Hence, in the foregoing example, all active electrodes on the lead that are used to delivery a blocking waveform have a common polarity, namely the polarity of an anode, while the housing of the IPG is set to have a cathode polarity. Optionally, in alternative embodiments, most of the electrodes on the lead may have an anodic polarity, but there may be at least one cathode electrode on the lead, as opposed to only using the IPG housing as the cathode for blocking.

The memory 158 includes a manager module 183 that directs the controller 151 to manage the delivering operations to do one or more of: time delivery of the SCS excitation and blocking waveforms to at least partially overlap; adjust a timing of the blocking waveform based on a propagation speed at which action potentials propagate along the nerve fibers; and/or shape the blocking waveform with at least one pulse having a trailing edge with a non-zero slope. The excitation, blocking and manager modules 181-183 direct the controller 151 to perform various operations.

For example, the manager module 183 may time a delivery of pulses in the blocking waveform to at least one of selectively prevent excitation or impede action potential propagation in nerve fibers within the DR corresponding to the nerve fiber NTR. The blocking module 182 may generate multiple blocking pulses in the blocking waveform in connection with a single SCS pulse in the SCS excitation waveform, the blocking pulses timed to at least partially overlap the SCS pulse. The manager module 183 may time the blocking pulse to start earlier or end later than the SCS pulse. The excitation and blocking modules 181 and 182 may also deliver recharge pulses following the excitation and blocking waveforms, the recharge pulses configured to at least partially neutralize a charge remaining on the stimulating electrodes following delivery of the excitation and blocking waveforms.

In some embodiments, the controller 151 measures an action potential (AP) signal to obtain AP activity data for the nerve fiber NTR and derives the propagation speed of the nerve fiber NTR based on the AP activity data. The manager module 183 adjusts the timing of the blocking waveform based on the propagation speed at which action potentials propagate along the nerve fibers. The pulse generating circuitry 152 may include first and second current sources 185 and 186 that independently deliver the SCS excitation waveform and blocking waveform, respectively. The pulse generating circuitry 152 shapes one or more pulses of the blocking waveform to have a tapered trailing average with a nonzero slope when transitioning between high and low levels. The controller 151 couples the blocking electrode to an anode of the pulse generator, such that the blocking electrode forms an anodic electrode when delivering the blocking waveform. The controller 151 couples the housing of the IPG to a cathode of the pulse generator such that the housing forms a cathode electrode when delivering the blocking waveform.

A controller device 160 may be implemented to charge/recharge the battery 154 of the IPG 150 (although a separate recharging device could alternatively be employed) and to program the IPG 150 on the pulse specifications while implanted within the patient. Although, in alternative embodiments separate programmer devices may be employed for charging and/or programming the NS system 100. The controller device 160 may be a processor-based system that possesses wireless communication capabilities. Software may be stored within a non-transitory memory of the controller device 160, which may be executed by the processor to control the various operations of the controller device 160. A “wand” 165 may be electrically connected to the controller device 160 through suitable electrical connectors (not shown). The electrical connectors may be electrically connected to a telemetry component 166 (e.g., inductor coil, RF transceiver) at the distal end of wand 165 through respective wires (not shown) allowing bi-directional communication with the IPG 150. Optionally, in some embodiments, the wand 165 may comprise one or more temperature sensors for use during charging operations.

The user may initiate communication with the IPG 150 by placing the wand 165 proximate to the NS system 100. Preferably, the placement of the wand 165 allows the telemetry system of the wand 165 to be aligned with the far-field and/or near field communication circuitry 155 of the IPG 150. The controller device 160 preferably provides one or more user interfaces 168 (e.g., touchscreen, keyboard, mouse, buttons, or the like) allowing the user to operate the IPG 150. The controller device 160 may be controlled by the user (e.g., doctor, clinician) through the user interface 168 thereby allowing the user to interact with the IPG 150. The user interface 168 may permit the user to move electrical stimulation along and/or across one or more of the lead(s) 110 using different stimulation electrode 121 combinations, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME,” which is expressly incorporated herein by reference.

Also, the controller device 160 may permit operation of the IPG 150 according to one or more therapies to treat the patient. Each therapy and blocking waveform may include one or more sets of stimulation or blocking parameters of the pulse including pulse amplitude, pulse width, pulse frequency or inter-pulse period, pulse repetition parameter (e.g., number of times for a given pulse to be repeated for respective stimset during execution of program), waveform shape (e.g., biphasic pulses, monophasic pulses), etc. The IPG 150 modifies its internal parameters in response to the control signals from the controller device 160 to vary the characteristics of the stimulation and blocking pulses transmitted through the lead 110 to the tissue of the patient. NS systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are expressly incorporated herein by reference.

FIGS. 2D and 2E illustrate an isometric view and a cross-section, respectively, of a paddle lead 230 formed in accordance with one embodiment. The paddle lead 230 includes a lead body 232 having a distal end 234, a proximal end 236, and a central axis 238 extending there between. The central axis 238 extends generally along a geometric center of a cross-section of the paddle lead 230. The paddle lead 230 includes a cable 240 that extends from the proximal end 236. The cable 240 includes conductive pathways (e.g., wires) that extend from the lead body 232 to a pulse generator (not shown). The lead body 232 also includes opposite first and second sides 242 and 244 that extend between the distal and proximal ends 234, 236. The paddle lead 230 also includes longitudinal edges 248, 250 that extend along a length (e.g., a greatest dimension) of the lead body 232. The longitudinal edges 248, 250 may extend generally along or parallel to the central axis 238. The longitudinal edges 248, 250 may also join the first and second sides 242, 244.

The paddle lead 230 also includes a plurality of electrodes 246 that are disposed along the first side 242 and are configured to provide neurostimulation therapy and/or blocking waveforms. In the illustrated embodiment, the electrodes 246 form an array that includes a 5×4 grid of electrodes 246 in which the electrodes 246 are substantially evenly distributed in rows and columns relative to the central axis 238. The lead includes an array of electrodes 121, 246 including the blocking electrode. The blocking electrode has an anodic polarity when delivering the blocking waveform in order that the lead excludes any electrodes with a cathodic polarity in connection with delivering the blocking waveform. In alternative embodiments, the electrodes 246 may form a single row or column that extends along the central axis 238 and are spaced apart from each other. The second side 244, when the paddle lead is disposed in the epidural space, may interface with an anatomical structure (e.g., bone, ligament, or other portions of the spinal column). Although not indicated, the lead body 232 may have a central portion and first and second wing portions like the lead body 202. The lead body 232 also has a length, width, and thickness.

In particular embodiments, the characteristics of the materials used to form the lead bodies 202, 232 and/or the dimensions (e.g., thickness) of the lead bodies 202, 232 permit the lead bodies 202, 232 to be readily flexible or conformable (e.g., supple) when a designated amount of fluid pressure provided through the dura is experienced. The lead body 202, 232 may include one or more biocompatible materials. The materials may be electrically insulating (e.g., dielectric materials). Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane.

Optionally, at least some of the arrays or patterns of electrodes may be irregular. For instance, one row (or column) of electrodes may have different separation distances between adjacent electrodes and an adjacent row (or column) may also have different separation distances between adjacent electrodes. The patterns for each of the rows (or columns) may be different. As one example of differently patterned rows or columns, a first row or column may have five electrodes in which the first and second electrodes are separated by X, the second and third electrodes are separated by 2X, the third and fourth electrodes are separated by 0.9X, and the fourth and fifth electrodes are separated by 0.8X.

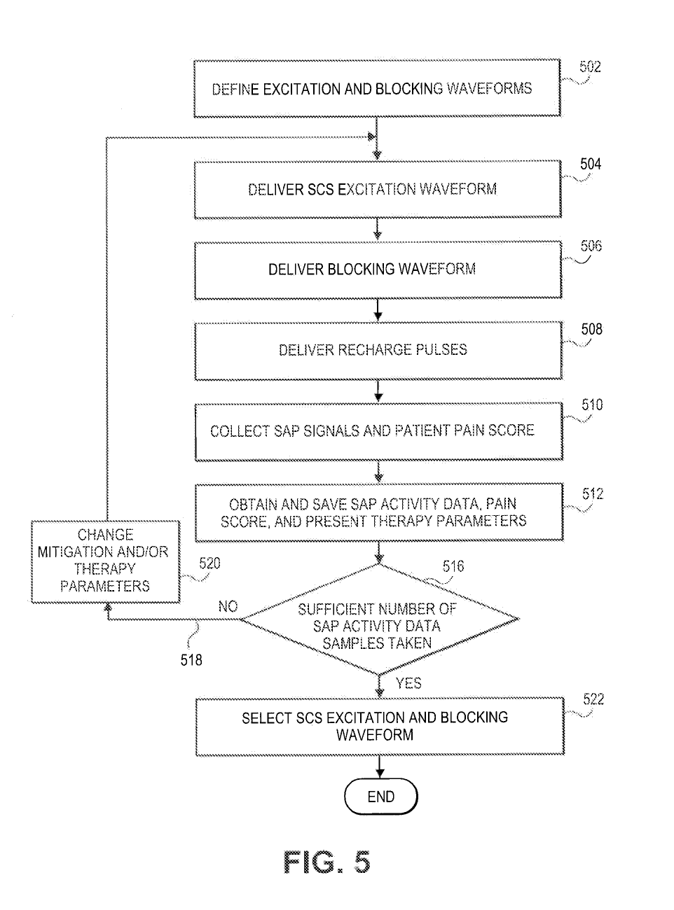

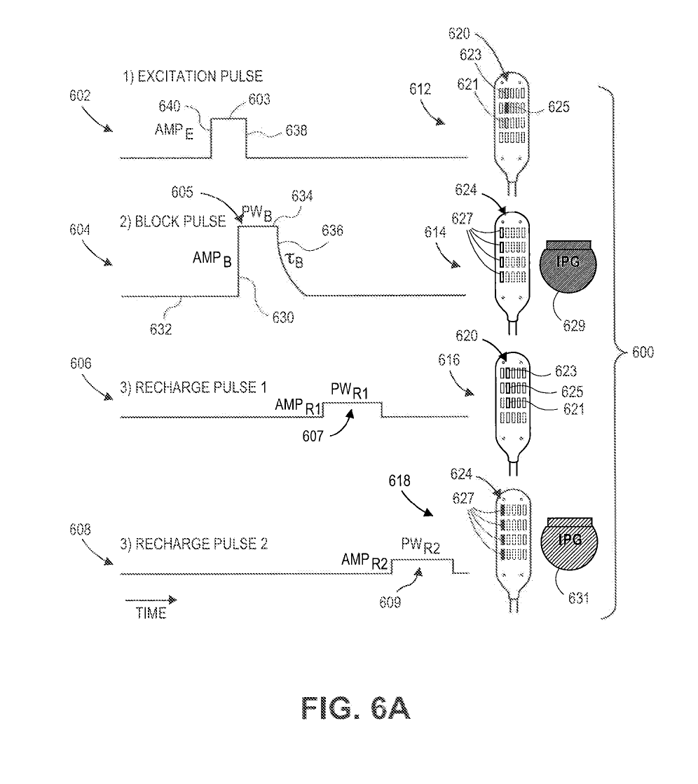

FIG. 5 illustrates a process to mitigate excitation of non-target regions of nerve fibers during spinal cord stimulation of nervous tissue of a patient. The operations of the Figures may be implemented by one or more processors, such as within an implantable pulse generator, external programmer, another external device and the like. The IPG, external programmer or other external device are coupled to a lead having at least one stimulation electrode that is implanted at a target position proximate to nervous tissue of interest.

At 502, the method defines one or more SCS excitation waveforms and one or more blocking waveforms. As noted above, the SCS stimulation and blocking waveforms are defined by one or more parameters forming a therapy parameter set (TPS) and a mitigation parameter set (MPS). Examples of therapy and mitigation parameters include, but are not limited to pulse amplitude, pulse width, interpulse delay, number of pulses per burst, pulse frequency, burst frequency, etc. The excitation and blocking waveforms are defined by amplitude, pulse width, waveform shape, and electrode configuration, which are selected to achieve a desired clinical efficacy, such as coverage of painful areas. The TPS is defined such that the stimulation waveform shape/morphology is configured to excite at least one of A-beta fibers, A-delta fibers, and/or C-fibers at the nerve fiber target region within the dorsal column. For example, the TPS may cause the stimulation waveform to exhibit a shape/morphology that excites A-beta fibers sufficiently to induce notable paresthesia. The MPS is defined such that the blocking waveform shape/morphology at least partially induces hyperpolarization in a nerve fiber non-target region (e.g. within the dorsal root or dorsal root ganglia). The shape of the blocking pulses within the blocking waveform may be rectangular, trapezoidal, or some other shape. A trapezoidal pulse shape may be desirable in order to help prevent excitation at the end of the blocking pulse (“anodal break excitation”). By way of example, the blocking waveform may have a trapezoidal shape or another non-rectangular shape. As a further example, the blocking waveform may have one or more pulses with a shape/morphology having a tapered trailing edge with a nonzero slope when transitioning from a high-level to a low level.

At 504, the method delivers the SCS excitation waveform from one or more electrodes.

At 506, the method delivers the blocking waveform. The method manages the delivery of the SCS and blocking waveforms in order to time delivery of the SCS excitation and blocking waveforms to at least partially overlap one another. The method times a delivery of pulses in the blocking waveform to selectively prevent excitation of the nerve fiber NTR. The method times delivery of pulses in the blocking waveform to impede propagation of an action potential along the nerve fibers within the dorsal root in the afferent direction (e.g., toward the brain), such as in an area corresponding to the nerve fiber NTR. By impeding propagation of action potentials in the afferent direction, the blocking waveform may reduce side effects of the SCS excitation waveform.

Optionally, based on the MPS, the delivery operation at 506 may generate multiple successive blocking pulses within the blocking waveform to be delivered concurrently with a single SCS pulse in the SCS excitation waveform. The blocking pulses are timed to at least partially overlap the SCS pulse. For example, a blocking pulse may begin before an SCS pulse but continue until overlapping the SCS pulse. Optionally, the blocking pulse may begin at an intermediate point along the SCS pulse and continue after the SCS pulse terminates. Accordingly, the SCS pulse and blocking pulse may be timed such that the delivery operations at 504 and 506 cause the blocking pulse to start earlier or later than the corresponding SCS pulse.

Additionally or alternatively, the blocking waveform may be timed to induce hyperpolarization of the nerve fiber non-target region within the dorsal root at a common point in time at which an action potential is initiated in the nerve fiber target region within the dorsal column.

The timing of the excitation and blocking waveforms may be varied. For example, an individual blocking pulse may be initiated first, followed immediately or after a short delay by initiation of an excitation pulse, where a tail portion of the blocking pulse overlaps at least an initial portion of the excitation pulse. When initiating a blocking pulse before an excitation pulse, the blocking pulse provides preparatory hyperpolarization of DR fibers to prevent action potential initiation or impede action potential propagation generated by the subsequent excitation pulse. Optionally, the excitation and blocking pulses may be timed to overlap substantially, where one of the excitation and blocking pulse is initiated only slightly before the other of the excitation and blocking pulse.

At 508, the method delivers one or more recharge pulses to maintain charge balance in connection with the SCS excitation and blocking pulses, respectively. When the excitation, blocking, and recharge pulses are delivered independently in time, a single voltage or current source system may be used. Alternatively, when delivering excitation and blocking pulses in at least a partially overlapping manner, a first voltage or current source is utilized in connection with the excitation pulses, while a second voltage or current source is utilized in connection with the blocking pulses. Alternatively, the recharge pulses may be applied in combination with the excitation and blocking pulses (e.g., interleaved or concurrently). A system with two or more voltage or current sources may be utilized to concurrently apply the stimulation, blocking, and/or recharges pulses with differing stimulation parameters.

The SCS excitation and blocking waveforms delivered at 504, 506 are delivered independently. As one example, one potential benefit of independent SCS excitation and blocking waveforms is that the therapy parameter set (e.g. amplitude, pulse width, and waveform shape), defining the SCS excitation waveform, may differ from the mitigation parameter set, defining the blocking waveform. Utilizing separate TPS and MPS values affords the opportunity to utilize higher intensity blocking pulses (as compared to an intensity of the excitation pulses). In some instances, it may be desirable to utilize higher intensity blocking pulses in order to afford a desired level of hyperpolarization the nerve fibers in the non-target region (e.g., DR fibers), without affecting the therapy parameter set.

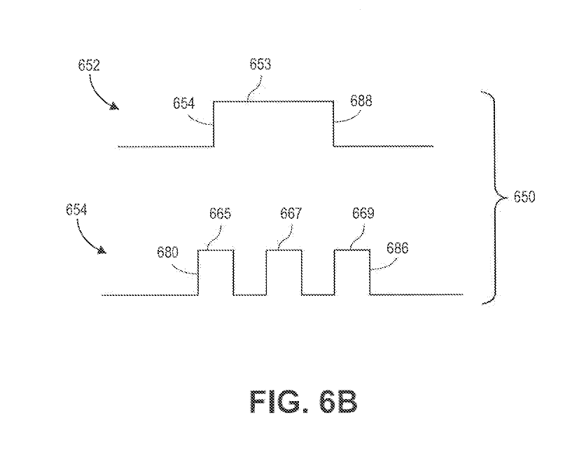

FIG. 6A illustrates examples of a stimulation and blocking pulse delivery timing provided in accordance with an embodiment herein, in order to afford anodal blocking of propagation of action potentials within the dorsal root. The timing diagram 600 illustrates an excitation waveform 602, a blocking waveform 604, an excitation recharge waveform 606 and a blocking recharge waveform 608. Examples of electrode configurations 612-618 are illustrated next to each of the waveforms 602-608, respectively. The electrode configurations 612-618 illustrate the electrodes that are activated as anodic or cathodic electrodes to deliver the waveforms 602-608. For example, electrodes within a central column 620 are utilized to deliver the excitation waveform 602, with a pair of electrodes 621 and 623 designated as anodal electrodes, with a single electrode 625 designated as a cathodic electrode. The cathodic electrode 625 is positioned between the anodal electrodes 621, 623, that collectively deliver the excitation waveform 602. The excitation waveform 602 in the example of FIG. 6A includes a single excitation pulse having an amplitude AMP_{E} and a pulse width PW_{E}.

As a further example, the electrode configurations 614 to deliver the blocking waveform 604 utilizes electrode within an outer column 624 to deliver the blocking waveform 604, with all of the electrodes 627 in the column 624 designated as anodal electrodes. A housing of the IPG is used as the cathodic electrode 629. The anodal electrodes 627 are arranged in a column located proximate to the nerve fiber non-target region within the dorsal root, and are collectively utilized to deliver the blocking waveform 604. The excitation waveform 602 includes a single blocking pulse having an amplitude AMP_{B} and a pulse width PW_{B}. The blocking pulse includes a leading edge 630 when transitioning from a low level 632 to a high level 634. The blocking pulse also includes a trailing edge 636 when transitioning from the high-level 634 back to the low level 632.

The trailing edge 634 includes an anti-break excitation shape in order to provide an overall shape or morphology for the blocking pulse that prevents excitation at an end of the blocking pulse (also referred to as “anodal break excitation”). As one example, the anti-break excitation shape of the trailing edge 634 represents a linear or nonlinear of line transitioning from the high-level 634 to the low level 632 while exhibiting a nonzero slope. In the example of FIG. 6, the trailing edge 634 includes a curved shape, such as resembling a capacitor discharge curve where the curve of the shape corresponds to a decay time constant T_{B} associated with an RC discharging circuit (e.g., T_{B}=t/RC; where t corresponds to the discharged time, R correspond to the circuit resistance and C corresponds to the capacitance of the capacitor being discharged). Optionally, the trailing edge 634 may have other shapes, such as a straight line (with a nonzero negative slope), a stair stepped shape, as well as a shape defined by second order or higher order polynomials.

As explained herein, the excitation and blocking pulses 603 and 605 overlap in time. In the example of FIG. 6A, the excitation pulse 603 begins before but continues in an overlapping manner with the blocking pulse 605 such that the leading edge 630 of the blocking pulse 605 occurs before the trailing edge 638 of the excitation pulse 603. Optionally, the timing of the excitation and blocking pulses 603 and 605 may be shifted temporally forward or backward with respect to one another, such as based on the conduction velocity or propagation speed of the fibers. For example, leading edges 640 and 630 of the excitation and blocking pulses 603 and 605, respectively, may occur at the same time. Additionally or alternatively, trailing edges 638 and 636 of the excitation and blocking pulses 603 and 605 may occur at the same time. As another example, the blocking pulse 605 may begin before the excitation pulse 603 but continue in an overlapping manner.

The electrode configuration 616 may be utilized to deliver an excitation recharge pulse 606 to offset the charge introduced by the excitation waveform 602. The electrode configuration 616 utilizes electrodes in the central column 620 where the electrodes 621 and 623 are defined as cathodic electrodes and the electrode 625 defined as an anodic electrodes. The cathodic electrodes 621, 623 are located on either side of the anodic electrode 625, thereby introducing a charge pulse 607 to offset the charge introduced by excitation pulse 603. The recharge pulse 607 includes an amplitude AMP_{R1} and pulse width PW_{R1} that differ from the amplitude and pulse width of the excitation pulse 603. Optionally, the recharge pulse 607 may exhibit a common total energy (e.g. the area under the curve) as the energy introduced by the excitation pulse 603.

The electrode configuration 618 may be utilized to deliver a blocking recharge pulse 609 to offset the charge introduced by the blocking waveform 604. The electrode configuration 618 utilizes electrodes in the peripheral column 624, where all of the electrodes 627 are defined as cathodic electrodes, and the housing of the IPG as signed as an anodic electrode 631. The cathodic electrodes 627 introduce a blocking recharge pulse 609 to offset the charge introduced by blocking pulse 605. The recharge pulse 609 includes an amplitude AMP_{R2} and pulse width PW_{R2} that differ from the amplitude and pulse width of the excitation pulse 603. Optionally, the recharge pulse 609 may exhibit a common total energy (e.g. the area under the curve) as the energy introduced by the blocking pulse 605.

The timing of the recharge pulses 607 and 609 may be adjusted to partially overlap one another or be separate and discrete from one another with a timing delay there between. The recharge pulses 607 and 609 may be timed to overlap the excitation pulse 603 and/or the blocking pulse 605.

FIG. 6B illustrates an example of a stimulation and blocking pulse delivery timing provided in accordance with an embodiment herein. The timing diagram 650 illustrates an excitation waveform 652 and a blocking waveform 654. The blocking waveform 654 includes a series of successive blocking pulses 665, 667 and 669 that at least partially overlap the excitation pulse 653. In the example of FIG. 6B, the excitation pulse 665 includes a leading-edge 680 that precedes a leading-edge 654 of the excitation pulse 653. Further, a trailing edge 686 of a final blocking pulse 669 follows the trailing edge 688 of the excitation pulse 653. While the blocking pulses 665, 667 and 669 are illustrated as rectangular shapes, as explained herein nonrectangular pulses may be utilized.

Optionally, the excitation and blocking waveforms 652 and 654 may both include more than one excitation and blocking pulse 653 and 655, respectively. As one example, the excitation and blocking waveforms 652 and 654 may include an equal number of pulses. Alternatively, the excitation waveform 653 may include more or fewer pulses than the blocking waveform 654. As another example, multiple blocking pulses 655 may occur during the single excitation pulse 653.

Returning to FIG. 5, the method may repeat the operations at 304-308 indefinitely, without continuing on to the subsequent operations illustrated in FIG. 3. Alternatively, the method may continue from 308 to 310, where information is collected in connection with determining changes in the therapy parameter set and/or mitigation parameter set.

Throughout the embodiments described herein, the same electrodes may be used for sensing and stimulation. Alternatively, one group of electrodes may be used for sensing, while a different group of electrodes are used for stimulation. For example, the sensing electrodes may be spaced apart along the lead from the stimulation electrodes. Optionally, the sensing electrodes may be provided on a separate lead unique and distinct from the lead that includes the stimulation electrodes. For example, an SCS lead may be positioned along the spinal column at a desired location in order to deliver therapy at one or more stimulation sites of interest, while a separate sensing lead is provided. As one example, electrodes proximate to the dorsal column may be used for stimulation, while separate electrodes proximate to the dorsal root ganglion (DRG) or dorsal root (DR) are used for sensing. As a further option, sensing electrodes may be located remote from the DRG or DR, such as within the torso of the body and/or along the extremities of the patient, such as within the arms and legs. Optionally, the stimulation waveform may be delivered at electrodes proximate both of the dorsal column and the DRG, while sensing is performed at the DRG or DR.

At 510, the method senses SAP signals and collects the SAP signals for a data collection window. The SAP signals are collected at one or more electrodes located proximate to the nerve fibers at the non-target region near the dorsal root. The SAP signals are indicative of the sensory action potential experienced by nervous tissue of interest at the nerve fibers in the non-target region (e.g. proximate to the dorsal root). The SAP signals may represent an evoked compound action potential (ECAP) signal that is invoked by the SCS excitation waveform. A level of activity in the SAP signals may be indicative of an effectiveness of a blocking waveform. For example, when a blocking waveform is effective, the method would expect the SAP signals to exhibit relatively low activity (e.g. amplitude, energy, etc.), such as compared to a baseline. Alternatively, when the blocking waveform is ineffective, the SAP signals may exhibit relatively high activity, such as compared to a baseline.

The SAP signals, collected over a single data collection window, represent an SAP sample for a single time interval, where the SAP sample is indicative of a responsiveness of the fibers of interest (C-fibers, A-beta fibers and/or A-delta fibers). For example, the responsiveness may correspond to a predetermined amount of touch, pressure, and external temperature source, or any other external input that would otherwise cause activity within the fibers of interest. The SAP signals are saved as an SAP sample.

At 510, the patient may also enter a pain score to indicate an amount/degree of pain experienced by the patient relative to a predetermined pain index. For example, a blocking waveform may not be effective in suppressing pain or other sensations introduced by the SCS excitation waveform into nerve fibers at a non-target region. Accordingly, the patient may enter a pain score representative of pain experienced even while a blocking waveform is utilized, thereby indicating that a different blocking waveform may be warranted. Optionally, the patient may enter a side effect score representative of side effects being experienced.

At 512, the method analyzes the SAP signal (e.g., the SAP sample) and the patient entered pain score (if provided) to obtain activity data associated with the mitigation parameter set used to define the blocking waveform. The activity data corresponds to activity for the nerve fibers in the non-target region near the dorsal root. The analysis at 512 may be repeated numerous times to obtain a collection of activity data associated with multiple MPS for different blocking waveforms. In the embodiment illustrated in FIG. 5, the operation at 512 may be implemented during each iteration through the operation at 508-520. Optionally, the operation at 512 may be implemented once after an entire collection of activity data is obtained from a predetermined number of iterations through the operations at 508-520 for the group or multiple different combinations of mitigation parameter sets that define different blocking waveforms.

At 512, the method also saves the pain score, and activity data along with the values for the corresponding mitigation parameter set, such as in a memory of the IPG, external programmer or other external device. The activity data, pain scores and the associated mitigation parameter set are saved, over time, thereby developing a mitigation/sensitivity history for the patient. The mitigation/sensitivity history indicates, among other things, a degree to which certain blocking waveforms inhibit sensory action potentials along conduction nerve fibers in a non-target region.

At 516, the method determines whether a sufficient number of SAP samples and/or patient pain scores have been collected (and analyzed). When a sufficient number of SAP samples and/or patient pain scores have been collected, flow moves to 522. When it is determined that additional SAP samples should be collected, flow moves along 518 to 520.

At 520, the method changes a value for one or more of the parameters within the mitigation parameter set. The change at 520 may be performed in a predetermined systematic stepwise manner. For example, each parameter within the mitigation parameter set may be incrementally adjusted by a select amount during separate iterations through the operations at 508-516. As an example, during iterations 1-3, the method may only change the amplitude of the blocking waveform between low, medium and high amplitudes, while maintaining constant all other parameters within the MPS. After cycling through each of the pulse amplitudes of interest, the pulse amplitude may be reset to the low level for iterations 4-6, during which the pulse width is changed from short to medium to long. During iterations 7-9, the pulse amplitude may be set to the medium level, while the pulse width is again changed from short to medium to long, while all other parameters are maintained constant. The foregoing process may be repeated until each, or at least a select portion, of the potential permutations and combinations of levels for the parameters are used during the operations at 508-516 to form the group of MPS for which the collection of activity data is accumulated.

Alternatively or additionally, not all permutations and combinations of parameter levels may be used. For example, a physician or other user may select (and/or program) individual MPS of interest to be tested as the group of MPS. For example, the operations at 508-516 may only be repeated for 5 to 10 or 20 different MPS, even though many more permutations and combinations of levels for the various parameters exist. The change performed at 520 may be based on pre-stored settings or may represent an input from a physician or other user during operation.

Optionally, the therapy parameter set used to define the SCS excitation waveform may also be varied during iterations through 504-516. Optionally, the amount of change (for SCS excitation and/or blocking) during each iteration through 520 may vary, such as with larger step changes made during initial iterations and smaller step changes made during later iterations. Optionally, the amount of change at 520 may be based on a difference between the activity data and the threshold. For example, when the activity data substantially exceeds the threshold, larger changes may be applied to one or more parameters at 520. As the difference between the activity data and threshold decreases, the incremental change in the one or more parameters is changed by similarly/proportionally decreasing amounts. Following 520, flow returns to 508.

The operations at 508-516 may build a database, file, or generally a pain-activity data relation corresponding to a relation between various mitigation parameter sets and therapy parameter sets.

At 522, the method selects a candidate MPS from the multiple or group of MPS based on one or more criteria of interest. For example, when the criteria of interest represents a threshold or predetermined range for the activity data, the candidate MPS may be selected as the MPS that resulted in activity data that satisfy the threshold or predetermined range. For example, when the criteria of interest represents sensory activity, at 522, the method may identify the SAP sample for which the lowest or smallest amount of activity data was identified, thereby indicating that the blocking waveform is effective. The method cross references SAP sample, that exhibits the lowest or smallest amount of activity data, to the corresponding mitigation parameter set which is designated as the candidate MPS. As one example, the selection at 522 may seek to optimize the candidate MPS to define as a blocking waveform that affords an SAP activity below a threshold or within a range, collectively referred to as a result of interest. Once a candidate MPS is selected, the candidate MPS is used for subsequent blocking waveforms for a period of time, for example until it becomes desirable to repeat the process of FIG. 5 to determine a new candidate MPS.

The operations at 508-520 are iteratively repeated to form a feedback loop in which the therapy parameter set is continuously updated until obtaining a blocking waveform that inhibits action potentials.

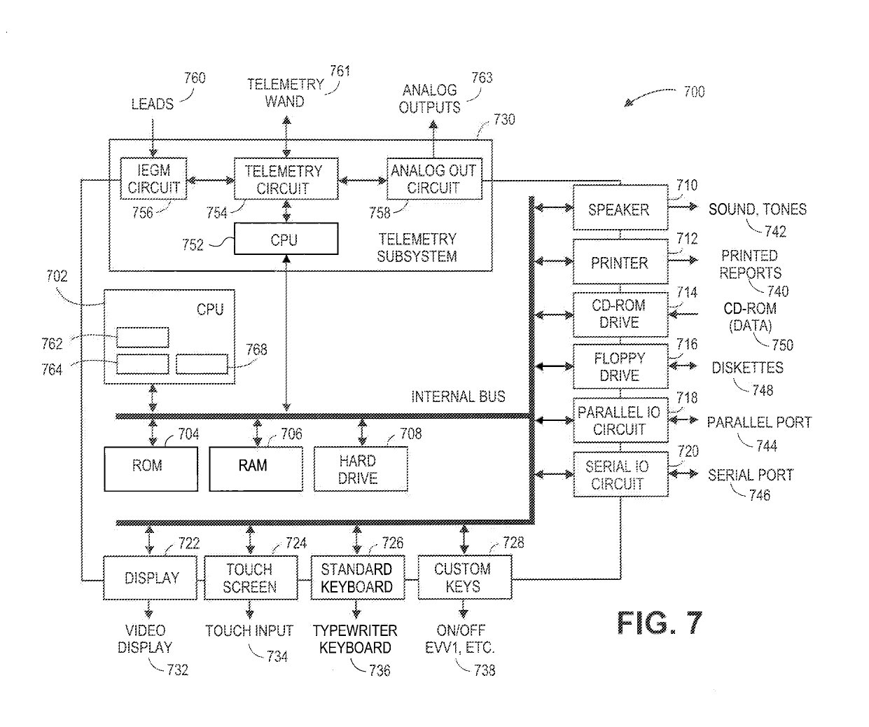

FIG. 7 illustrates a functional block diagram of an embodiment of an electronic control unit (ECU) 700 that is operated in accordance with the processes described herein to analyze SAP signals and to interface with one or more IPGs and/or leads with electrodes positioned at stimulation sites to deliver coupled tonic/burst therapies and/or sense sensory action potential signals. The ECU 700 may be a workstation, a portable computer, a PDA, a cell phone and the like. The ECU 700 includes an internal bus that connects/interfaces with a Central Processing Unit (CPU) 702, ROM 704, RAM 706, a hard drive 708, the speaker 710, a printer 712, a CD-ROM drive 714, a floppy drive 716, a parallel I/O circuit 718, a serial I/O circuit 720, the display 722, a touch screen 724, a standard keyboard connection 726, custom keys 728, and a telemetry subsystem 730. The internal bus is an address/data bus that transfers information between the various components described herein. The hard drive 708 may store operational programs as well as data, such as waveform templates and detection thresholds.

The CPU 702 typically includes a microprocessor, a microcontroller, or equivalent control circuitry, and may interface with an IPG and/or lead. The CPU 702 may include RAM or ROM memory, logic and timing circuitry, state machine circuitry, and I/O circuitry to interface with the IPG and/or lead. The display 722 (e.g., may be connected to the video display 732). The touch screen 724 may display graphic information relating to the CNS 110. The display 722 displays various information related to the processes described herein. The touch screen 724 accepts a user's touch input 734 when selections are made. The keyboard 726 (e.g., a typewriter keyboard 736) allows the user to enter data to the displayed fields, as well as interface with the telemetry subsystem 730. Furthermore, custom keys 728 turn on/off 738 (e.g., EVVI) the ECU 700. The printer 712 prints copies of reports 740 for a physician to review or to be placed in a patient file, and speaker 710 provides an audible warning (e.g., sounds and tones 742) to the user. The parallel I/O circuit 718 interfaces with a parallel port 744. The serial I/O circuit 720 interfaces with a serial port 746. The floppy drive 716 accepts diskettes 748. Optionally, the floppy drive 716 may include a USB port or other interface capable of communicating with a USB device such as a memory stick. The CD-ROM drive 714 accepts CD ROMs 750.

The CPU 702 may perform the operations discussed above in connection with FIG. 5 may perform the operations discussed above in connection with FIG. 5.

The CPU 702 also includes an SAP analysis circuit module 768 that receives sensed SAP signals from at least one electrode on the lead, and analyzes the SAP signals to identify a sensory action potential (SAP) component of the signals. For example, the SAP analysis circuit module 768 may determine an amount to adjust the blocking waveform. The SAP analysis circuit module 768 may analyze a feature of interest from a morphology of the SAP component over time, count a number of occurrences of the feature of interest that occur within the SAP component over a predetermined duration, compare the number of occurrences to a prior number of occurrences, and determine an amount to adjust the relative timing of excitation and blocking pulses based on the comparing operation. The SAP analysis circuit module 768 may analyze the SAP component to determine SAP activity level data for a present/current coupled tonic/burst therapy. The SAP activity level data is saved in memory with the associated therapy parameters.

An excitation module 762 performs operations similar to the excitation module 181 to control delivery of SCS excitation waveforms to the excitation electrode(s). A blocking module 764 performs operations similar to the blocking module 182 to control delivery of blocking waveforms to a blocking electrode located proximate to the DR.

The telemetry subsystem 730 includes a central processing unit (CPU) 752 in electrical communication with a telemetry circuit 754, which communicates with both an SAP circuit 756 and an analog out circuit 758. The circuit 756 may be connected to leads 760. The circuit 756 may also be connected to implantable leads to receive and process SAP signals. Optionally, the SAP signals sensed by the leads may be collected by the CNS 110 and then transmitted, to the ECU 700, wirelessly to the telemetry subsystem 730 input.

The telemetry circuit 754 is connected to a telemetry wand 761. The analog out circuit 758 includes communication circuits to communicate with analog outputs 763. The ECU 700 may wirelessly communicate with the CNS 110 and utilize protocols, such as Bluetooth, GSM, infrared wireless LANs, HIPERLAN, 3G, satellite, as well as circuit and packet data protocols, and the like. Alternatively, a hard-wired connection may be used to connect the ECU 700 to the CNS 110.

One or more of the operations described above in connection with the methods may be performed using one or more processors. The different devices in the systems described herein may represent one or more processors, and two or more of these devices may include at least one of the same processors. In one embodiment, the operations described herein may represent actions performed when one or more processors (e.g., of the devices described herein) are hardwired to perform the methods or portions of the methods described herein, and/or when the processors (e.g., of the devices described herein) operate according to one or more software programs that are written by one or more persons of ordinary skill in the art to perform the operations described in connection with the methods.

The controller 160 may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the controllers may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The controllers and the controller device may execute a set of instructions that are stored in one or more storage elements, in order to process data. The storage elements may also store data or other information as desired or needed. The storage element may be in the form of an information source or a physical memory element within the controllers and the controller device. The set of instructions may include various commands that instruct the controllers and the controller device to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f), unless and until such claim limitations expressly use the phrase “means for” followed by a statement of function void of further structure.

1. A method to mitigate excitation of non-target regions of nerve fibers during spinal cord stimulation (SCS) of nervous tissue of a patient, the method comprising:
delivering an SCS excitation waveform to an excitation electrode located proximate to a dorsal column (DC), the SCS excitation waveform shaped to excite a nerve fiber target region (TR) within the DC;
delivering a blocking waveform to a blocking electrode located proximate to the DR, the blocking waveform shaped to at least partially induce hyperpolarization into a nerve fiber non-target region (NTR) within the DR; and
managing the delivering operations to at least one of:
i) time delivery of the SCS excitation and blocking waveforms to at least partially overlap;
ii) adjust a timing of the blocking waveform based on a propagation speed at which action potentials propagate along the nerve fibers; or
iii) shape the blocking waveform with at least one pulse having a trailing edge with a non-zero slope.

2. The method of claim 1, wherein the excitation electrode is positioned proximate to the nerve fiber TR within the DC, wherein the blocking electrode is positioned proximate to the nerve fiber NTR within the DR.

3. The method of claim 1, wherein the blocking waveform has at least one of a trapezoidal shape or non-rectangular shape.

4. The method of claim 1, wherein the blocking waveforms induce hyperpolarization into at least one of A-delta fibers, A-beta fibers or C-fibers within the NTR.

5. The method of claim 1, further comprising positioning the at least one blocking electrode proximate to at least one of the dorsal root or dorsal root ganglion.

6. The method of claim 1, wherein the managing operation includes timing a delivery of pulses in the blocking waveform to at least one of selectively prevent excitation or impede action potential propagation in nerve fibers within the DR corresponding to the nerve fiber NTR.

7. The method of claim 1, wherein the delivering operation includes generating multiple blocking pulses in the blocking waveform in connection with a single SCS pulse in the SCS excitation waveform, the blocking pulses timed to at least partially overlap the SCS pulse.

8. The method of claim 1, wherein the SCS excitation and blocking waveforms include an SCS pulse and blocking pulse, respectively, the delivery operation timing the blocking pulse to start earlier or end later than the SCS pulse.

9. The method of claim 1, wherein the blocking waveform is configured to impede action potential propagation in nerve fibers within the DR in the afferent direction, thereby reducing side effects of the excitation waveform.

10. The method of claim 1, wherein the blocking waveform is configured to hyperpolarize the nerve fibers in the DR near AP initiation.

11. The method of claim 1, further comprising delivering recharge pulses following the excitation and blocking waveforms, the recharge pulses configured to at least partially neutralize a charge remaining in the nerve fibers following delivery of the excitation and blocking waveforms.

12. The method of claim 1, further comprising:
measuring an action potential (AP) signal to obtain AP activity data for the nerve fiber NTR;
deriving the propagation speed of the nerve fiber NTR based on the AP activity data; and
adjusting the timing of the blocking waveform based on the propagation speed at which action potentials propagate along the nerve fibers.

13. A system to mitigate excitation of non-target regions of nerve fibers during spinal cord stimulation (SCS) of nervous tissue of a patient, the system comprising:
a lead having an excitation electrode configured to be located proximate to a dorsal column (DC) and having a blocking electrode configured to be located proximate to the DR;
a processor;
a pulse generator; and
memory storing program instructions accessible by the processor; wherein, responsive to execution of the program instructions, the processor:
delivering a SCS excitation waveform to the excitation electrode, the SCS excitation waveform shaped to excite a nerve fiber target region (TR) within the DC;
delivering a blocking waveform to a blocking electrode located proximate to the DR, the blocking waveform shaped to at least partially induce hyperpolarization into a nerve fiber non-target region (NTR) within the DR; and
managing the delivering operations to at least one of:
i) time delivery of the SCS excitation and blocking waveforms to at least partially overlap;
ii) adjust a timing of the blocking waveform based on a propagation speed at which action potentials propagate along the nerve fibers; or
iii) shape the blocking waveform with at least one pulse having a trailing edge with a non-zero slope.

14. The system of claim 13, wherein the excitation electrode is positioned proximate to the nerve fiber TR within the DC, wherein the blocking electrode is positioned proximate to the nerve fiber NTR within the DR.

15. The system of claim 13, wherein the pulse generator further comprises first and second current sources that independently deliver the SCS excitation waveform and blocking waveform, respectively.

16. The system of claim 13, wherein the pulse generator shapes a pulse of the blocking waveform to have a tapered trailing average with a nonzero slope when transitioning between high and low levels.

17. The system of claim 13, wherein the lead includes an array of electrodes including the excitation and blocking electrodes.

18. The system of claim 13, wherein processor couples the blocking electrode to an anode of the pulse generator, that the blocking electrode formed an anodic electrode when delivering the blocking waveform, the system further comprising an implantable medical device having a housing coupled to the lead, the processor coupling the housing of the IPG to a cathode of the pulse generator such that the housing forms a cathodic electrode when delivering the blocking waveform.

19. The system of claim 13, wherein the lead includes an array of electrodes including the blocking electrode, the blocking electrode having an anodic polarity when delivering the blocking waveform, the lead excluding any electrodes with a cathodic polarity in connection with delivering the blocking waveform.

20. The system of claim 13, wherein the managing operation performed by the processor includes timing a delivery of pulses in the blocking waveform to at least one of selectively prevent excitation or impede action potential propagation in nerve fibers within the DR corresponding to the nerve fiber NTR.
